(19) United States
(12) Patent Application Publication
(10) Pub. No.: US 2022/0355070 A1
(43) Pub. Date: Nov. 10, 2022

(54) MULTI-LUMEN CATHETERS

(71) Applicant: Boston Scientific Scimed, Inc., Maple Grove, MN (US)

(72) Inventors: Andrew PIC, Northboro, MA (US); Gerald FREDRICKSON, Westford, MA (US); Daniel CONGDON, Hudson, MA (US)

(73) Assignee: Boston Scientific Scimed, Inc., Maple Grove, MN (US)

(21) Appl. No.: 17/661,806

(22) Filed: May 3, 2022

Related U.S. Application Data
(60) Provisional application No. 63/183,738, filed on May 4, 2021.

(51) Int. Cl. A61M 25/00

(52) U.S. Cl. CPC A61M 25/0028; A61M 2025/0037

(57) ABSTRACT

A delivery device may comprise a sheath configured to be inserted into a body lumen of a patient. The sheath may have a proximal end, a distal end, a first channel extending within the sheath from the proximal end to the distal end, and a second channel extending within the sheath from the proximal end to the distal end. A collective cross-sectional area of the first channel and the second channel may be more than 50% of a total cross sectional area of the sheath. The device may also be comprised of a handle at the proximal end of the sheath, wherein the handle is configured to provide fluid communication between a first port and the first channel and a second port and the second channel.

### CROSS-REFERENCE TO RELATED APPLICATIONS

This application claims the benefit of priority from U.S. Provisional Application No. 63/183,738, filed on May 4, 2021, which is incorporated by reference herein in its entirety.

### TECHNICAL FIELD

Various aspects of the disclosure relate generally to medical devices for delivery of therapeutic agents, fluids, or medical tools. Examples of the disclosure relate to medical devices for endoscopic delivery of a pressurized fluid and multiple parts of a therapeutic agent to a target treatment site.

### BACKGROUND

There exists a need to deliver agents during a treatment/operation, and/or preoperatively and postoperatively, to target treatment sites within the body to protect those sites from tissue degradation, especially during or after endoscopic and open surgical procedures of the gastrointestinal (GI) tract. Examples of endoscopic and open surgical procedures of the GI tract include colonic resection, bariatric surgery, esophagectomy, gastric bypass, and sleeve gastrectomy, among others. Procedures may result in perforation, post-surgical leaks, or other wounds of the tract.

### SUMMARY

Aspects of the disclosure relate to, among other things, devices and methods to deliver agents to target treatment sites within the body during a treatment/operation, and/or preoperatively and postoperatively. Each of the aspects disclosed herein may include one or more of the features described in connection with any of the other disclosed aspects.

According to certain aspects of the disclosure, a delivery device may comprise a sheath having a proximal end, a distal end, a first channel extending within the sheath from the proximal end to the distal end, and a second channel extending within the sheath from the proximal end to the distal end. A collective cross-sectional area of the first channel and the second channel is more than 50% of a total cross sectional area of the sheath. The delivery device may also comprise a handle at the proximal end of the sheath. The handle may be configured to provide fluid communication between a first port and the first channel and a second port and the second channel.

The delivery device may include one or more of the following features. A first channel of the delivery device may be defined by (1) at least one first planar surface that extends from the proximal end to the distal end of the second channel, and (2) a first side opening that extends from the proximal end to the distal end of the second channel. A second channel may be similarly defined. The first and second channels may be separated by an inter-lumen wall. The side openings separate a first and a second flange. The first and second flanges may be configured to flex radially inward and radially outward. The delivery device may comprise a tube within the first channel, wherein a space is defined between the first planar surface and an exterior surface of the tube. The delivery device may contain an interior sheath and an exterior sheath surrounding the interior sheath, wherein an at-least partly annular space is defined between the exterior sheath and the interior sheath. The exterior sheath may be rotatable relative to the interior sheath. The exterior sheath and interior sheath may be coupled together by one or more connectors that partially or entirely extend from the proximal end to the distal end of the sheath between an internal surface of the exterior sheath and an external surface of the interior sheath. The interior sheath may be centered within the exterior sheath by one or more ribs between an internal surface of the exterior sheath and an exterior surface of the interior sheath, such that the at-least partly annular space may completely surround the interior sheath. The ribs may alternately protrude from the internal surface of the exterior sheath and the external surface of the interior sheath, such that the rotation of the interior sheath is limited to a predetermined angle. The interior sheath may define at least two channels separated by an inter-lumen wall that may extend from the proximal end to the distal end of the sheath. The delivery device may also comprise a third port of the handle is in fluid communication with the at-least partly annular channel. the handle may include a proximal handle piece and a distal handle piece, wherein the interior sheath may be fixed to the proximal handle piece, and the exterior sheath may be fixed to the distal handle piece, wherein a locking mechanism may couple the proximal handle piece to the distal handle piece such that the proximal handle piece may rotate relative to the distal handle piece and the interior sheath may rotate relative to the exterior sheath. The proximal handle piece may include a flange, the distal piece may include one or more protrusions proximal of the flange to couple the proximal handle piece to the distal handle piece, each protrusion may be integral with a corresponding flexible post, and each flexible post may be configured to bend to extend over the flange, permitting the protrusion to enter a space of the handle proximal to the flange. The delivery device may further comprise a third channel extending within the sheath from the proximal end to the distal end, wherein a collective cross-sectional area of the first channel, the second channel, and the third channel may be more than 75% of a total cross sectional area of the sheath.

According to another aspect of the disclosure, a delivery device may comprise an exterior sheath coupled to a handle; an interior sheath surrounded by the exterior sheath to define an at-least partly annular channel between the interior sheath and the exterior sheath, wherein the interior sheath is coupled to the handle; and a port that may be in fluid communication with the at-least partly annular lumen.

The delivery device may include one or more of the following features. An additional port may be attached to each of two channels of the interior sheath such that the additional ports may be in fluid connection with each respective channels of the interior sheath. The handle may comprise a proximal handle piece and a distal handle piece, wherein the interior sheath may be fixed to the proximal handle piece, and the exterior sheath may be fixed to the distal handle piece, wherein a locking mechanism may couple the proximal handle piece to the distal handle piece such that the proximal handle piece may rotate relative to the distal handle piece and the interior sheath may rotate relative to the exterior sheath. The proximal handle piece may include a flange, and the distal piece may include one or more protrusions proximal of the flange to couple the proximal handle piece to the distal handle piece.

According to another aspect of the disclosure, a delivery method may comprise placing a distal end of a delivery device proximate a treatment site and, at a same time, deliver a first part of an agent through a first channel of a sheath to proximate the treatment site; deliver a second part of the agent through a second channel of the sheath to proximate the treatment site; and deliver a pressurized fluid through a third channel of the sheath to proximate the treatment site, wherein the pressurized fluid mixes the first and second parts of the agent for delivery onto the treatment site. A collective cross-sectional area of the first, second, and third channels may be more than 25% of a total cross sectional area of the sheath

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 is a perspective view of a medical device, according to aspects of this disclosure;

FIG. 2 is a perspective cross-sectional view of a portion of a length of a sheath of the medical device of FIG. 1, according to aspects of this disclosure;

FIG. 2A is a perspective cross-sectional view of a portion of a length of the sheath of FIG. 2, with tubes inserted, according to aspects of this disclosure;

FIGS. 3 to 6 are perspective cross-sectional views of portions of lengths of sheaths, according to aspects of this disclosure;

FIG. 7 is a perspective view of a medical device, according to aspects of this disclosure; and

FIG. 7A is a perspective cross-sectional view of a portion of the medical device of FIG.7, according to aspects of this disclosure.

### DETAILED DESCRIPTION

Aspects of the disclosure include devices and methods for delivering agents, including multi-part agents, to a target tissue site within a subject (e.g., patient). Application of a multi-part agent at a target tissue site, for example, can protect those sites from further tissue degradation. Current devices and methods for delivery of such multi-part agents are limited. With the lack of effective treatment option and tools, there is a need to apply agents to target treatment sites to protect those sites from further tissue degradation. Certain prophylactic agents may require multiple parts to be able to cure or stay adhered to a target tissue. Many agents that are delivered may also potentially cure in the catheter unless a secondary means of curing is implemented, such as photosensitive cure (i.e. light cure). Catheters for delivering agents may also have significant clogging issues due to agents curing within them. In some aspects of the disclosure, a delivery device includes a multi-lumen tube that delivers a first part of an agent, a second part of an agent, and a pressurized fluid within first, second, and/or third lumens of the delivery device. The shape and/or cross-sectional area of the agent delivery lumens permits a sufficient volume of agent parts to be delivered to the site, without curing or clogging in the delivery lumens, for example. The pressurized fluid, for example gas, assists with mixing the agents distal of the delivery device, at the treatment site. The delivery device may be a catheter, scope (endoscope, bronchoscope, gastroscope, ureteroscope, duodenoscope, colonoscope, etc.), tube, or sheath, inserted into a body cavity or lumen, for example the GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, or bodily incision.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Reference will now be made in detail to examples of the disclosure described above and illustrated in the accompanying drawings. Wherever possible, the same reference numbers will be used throughout the drawings to refer to the same or like parts.

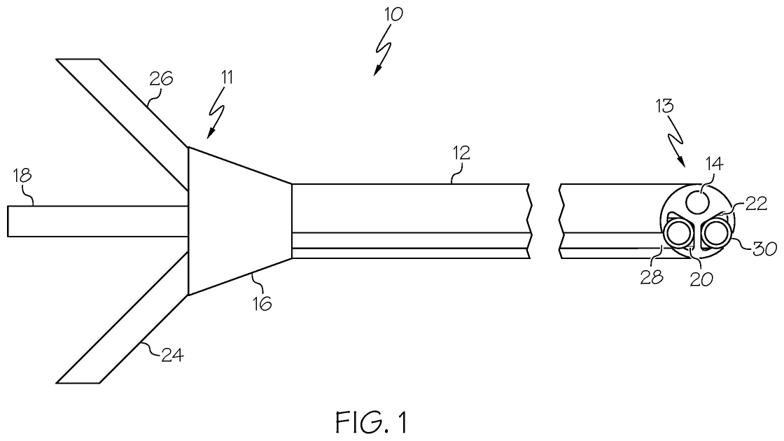

FIG. 1 shows a medical device 10 (e.g. agent delivery device) in accordance with an example of this disclosure. Medical device 10 includes a proximal end 11 and a distal end 13. A handle 16 and ports 18, 24, and 26 are at or adjacent to proximal end 11. A sheath 12 extends from a distal end of a handle 16 to a distal end 13 of device 10. Any of the structures of medical device 10 described herein can be made of biocompatible materials, including certain polymers, rubbers, plastics, and the like.

Still referring to FIG. 1, proximal ports 18, 24, and 26 permit access to the lumens (channels) of a sheath 12 (to be described further herein). Ports are configured to connect to sources of agents, gas, fluids, or the like, and further can provide access for devices to enter device 10 and insert into sheath 12. The sources of such agents, gases, or fluids can be any suitable sources known in the art. Such sources can include one or more agent containers, for example, syringes, canisters, etc. Alternatively, one or more devices, for example, catheters, snares, forceps, graspers, imaging devices, or any other endoscopic instrument known in the art, can be inserted through the ports and their respective sheath lumens. The connections between the sources of agent, gas, fluid, etc. and ports 18, 24, and 26 can be, for example, an adhesive, a heat shrink, a snap-fit connection, a threaded coupling, a crimping connection, a luer connection, and the like. The connection can be permanent or detachable. Each of the one or more agent containers can contain an agent component that is dispersible out of a respective lumen of the flexible sheath 12. Ports 18, 24, and 26 are in fluid connection with lumens within handle 16 (not shown) and sheath lumens.

Handle 16 is a junction for ports 18, 26, and 24 and sheath 12. In embodiments, handle 16 can include and/or surround the ports, enabling their access to and connection with the various sources of agent and devices known in the art. Handle 16 may be configured to be fixed to a distal end of each port 18, 24, and 26. Agent containers can be attached to a proximal end of one or more of ports 18, 24, and 26. These connections can be via, for example, an adhesive, a heat shrink, a snap-fit connection, a threaded coupling, a crimping connection, and the like. Handle 16 provides for the fluid communication between ports 18, 24, and 26, and the lumens of sheath 12 and also provides for access of devices from ports to the lumens of sheath 12. Handle 16 can include any actuators known in the art to control the supply of agent, gas, fluid, etc. from ports 18, 24, and 26 to sheath 12. These actuators can include triggers, buttons, switches, pneumatic controls, or other methods known in the art. In embodiments, device 10 does not include a handle, instead having ports 18, 24, and 26 lead to and communicate with the lumens of sheath 12, without any intervening structure.

Sheath 12 is a tube having sufficient length and flexibility to access sites within the body and traverse tortuous anatomy. Lumens 14, 20, and 22 extend through sheath 12 from the proximal end to the distal end of sheath 12. Lumens 14, 20, and 22 are open at a distal face of sheath 12, as shown in FIG. 1. Lumens 14, 20, and 22 are substantially parallel to each other, so that longitudinal axes of lumens 14, 20, and 22 are substantially parallel. Sheath 12 is not limited to contain three lumens 14, 20, and 22, as it can contain more or less lumens. Sheath 12 is connected to handle 16 at the proximal end of sheath 12, permitting access to lumens 14, 20, and 22 from ports 18, 24, and 26 through handle 16. In particular, port 18 permits access to lumen 14, port 26 to lumen 22, and port 24 to lumen 20.

Lumen 14 is a fully enclosed lumen (surrounded completely on all sides by portions of sheath 12) extending from the proximal end to the distal end of sheath 12. Lumen 14 has a circular cross-sectional shape, though it is not limited to such a shape, and is open at a distal face of sheath 12. There is a fluid connection between port 18, handle 16, and lumen 14. Multiple enclosed lumens 14 can be included in the cross-sectional area of the sheath and can have corresponding ports. The internal walls of lumen 14 could be lined with biocompatible materials, such as expanded polytetrafluoroethylene (ePTFE), to reduce friction for any agent or device being inserted through lumen 14. In embodiments, lumen 14 is configured for passage of a pressurized fluid (such as carbon dioxide gas), therethrough.

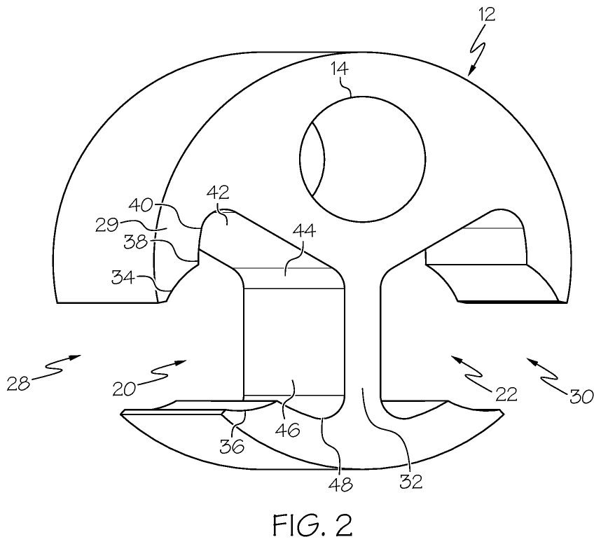

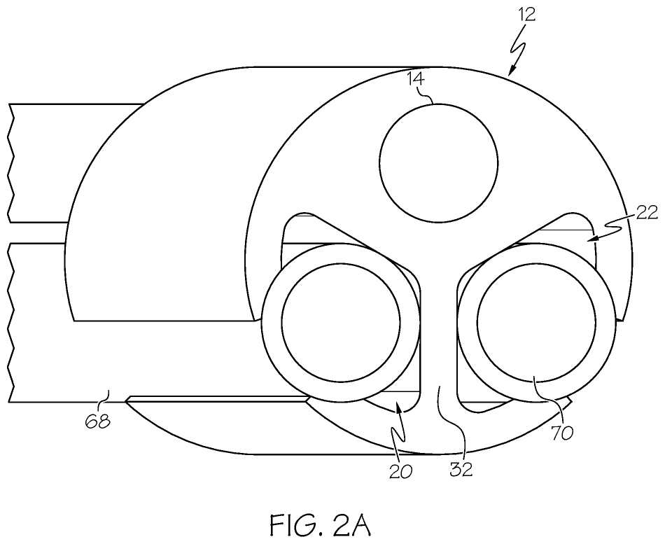

FIG. 2. shows a cross-section of a portion of a length of sheath 12 of FIG. 1. Sheath 12 may be the same or similar in cross-section along an entire length of sheath 12. Lumen 20 extends from the proximal end to the distal end of sheath 12.

There is a fluid connection between the corresponding port 24 and lumen 20. Lumen 20 is defined by planar surfaces 42, 46, curved surfaces 34, 36, 40, 44, and 48, and a side opening 28. The cross-sectional area of lumen 20 accounts for approximately one-fourth (¼) of the cross-sectional area of sheath 12, but that ratio is not limited, as there can be additional or fewer lumens and/or the sizes of lumens can be adjusted. The cross-sectional area of lumen 20 is maximized to accept a tube 68 (described further herein) of sufficient inner diameter that permits agent, for example, to pass therethrough without clogging the inside of tube 68, while leaving sufficient material of sheath 20 for sufficient structural integrity and passability along a tortuous body lumen. The collective cross-sectional areas of lumens 14, 20, and 22 is at least 50% of the total cross-sectional area of sheath 12, and in certain embodiments about two-thirds to at least about three-fourths of the total cross-sectional area of sheath 12.

Surfaces 34 and 40, and surfaces 36 and 48, along with a portion of the outer surface of sheath 12, define two flanges 29 and 31. Flanges 29 and 31 are located at the edges of opening 28. Flanges 29 and 31 are flexible, so that they can bend radially inward into lumen 28 and radially outward of sheath 12. Side opening 28, in combination with the flexibility of flanges 29 and 31, enables the cross sectional area of lumen 20 to expand for insertion and placement of tube 68. In addition or alternatively, the internal walls of lumen 20 could be lined with biocompatible materials, such as ePTFE, to reduce friction for a tube being inserted along the longitudinal axis of lumen 20. The side opening 28 is configured to enable a user to insert or remove a tube 68, as flanges 29, 31 flex in and out, respectively. Flanges 29, 31 extend the entire length of lumen 20. Surfaces 34 and 36 of flanges 29 and 31, respectively, can have a radius of curvature matching the curvature of tube 68, as shown in FIG. 2A.

Lumen 20 is defined by, among other surfaces, two planar surfaces 42, 46 oriented at an angle to each other and separated by curved surface 44. Planar surfaces 42, 46 create internal walls of the lumen 20 and extend the entire length of sheath 12. The planar shape of surfaces 42, 46 creates space between round tube 68 and those surfaces 42, 46, when tube 68 is inserted in lumen 20, as shown in FIG. 2A. Curved surfaces 40, 48 also create space between tube 68 and the surfaces defining lumen 20.

Lumen 22 is separated from lumen 20 by inter-lumen wall 32 and can be a mirror image of lumen 20. Lumen 22, however, is not limited to having the same geometry. For example, lumen 22 can vary in size and shape. There could be more than two lumens 20, 22, separated by additional inter-lumen walls. The number of lumens is dependent on, among other things, the cross-sectional area and material of sheath 12. For example, the material and walls of sheath 12 need to provide sufficient structural support to prevent the lumens and/or sheath from collapsing. Additionally, inter-lumen wall 32 should have sufficient thickness and rigidity to prevent lumens 20, 22 from changing shape, but in other embodiments, can have some flexibility to accommodate larger sized devices, including tubes, in at least one of lumens 20, 22.

As shown in FIG. 2A, tubes 68, 70 can each have a circular cross-sectional shape and thin walls to separate the space of lumens 20, 22 from the lumen of tube 68. The tubes 68, 70 can be alternative cross-sectional shapes, such as an oval, a triangle, or the exact cross sectional shape as the lumen to allow additional space within tube 68, 70 to reduce clogging. Additionally, different tubes can be inserted through lumens 20, 22. For example, a circular tube can be inserted through lumen 20, whereas a triangular tube can be inserted through lumen 22. Tubes 68, 70 can be inserted by the user at the corresponding port 24, 26, through the handle 16 or inserted into the lumen via the side-openings 28, 30. Alternatively, medical device 10 can be manufactured to include one or both tube 68, 70 preloaded into lumen 20, 22, respectively. Tubes 68, 70 can provide paths for an agent or a device to be delivered. Such devices can include a catheter, a snare, a grasper, forceps, or other endoscopic tools known in the art.

Aspects of the disclosure include methods of using device 10. To do so, the user first may attach sources of agent and/or pressurized fluid to one or more of ports 18, 24, and 26. Alternatively, such sources may be contained within handle 16 and already in fluid communication with lumens 14, 20, and 22. The user then may insert the distal end of sheath 12 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate the handle to permit agent and/or pressurized fluid flow through lumen 14 and/or tubes 68, 70. In an embodiment, the pressurized fluid flows through lumen 14, and two separate parts of an agent flow through tubes 68 and 70, respectively. The fluid and agent parts exit the distal ends of the lumen 14 and tubes 68, 70 to mix at the target treatment site and adhere to tissue. Alternatively, the user could use lumen 14, tubes 68, 70, or lumens 20, 22 to insert any other diagnostic and/or therapeutic tool.

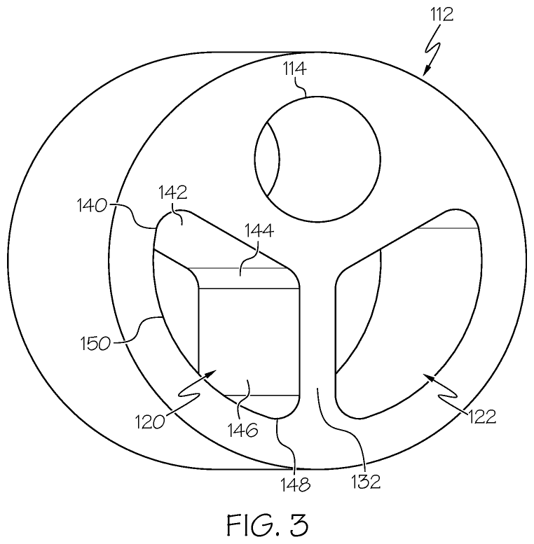

FIG. 3 depicts a cross-sectional view of a portion of a length of an exemplary embodiment of a sheath 112. Sheath 112 is identical to sheath 12 of FIGS. 1, 2, and 2A, except as described below. Sheath 112 is comprised of three closed lumens 114, 120, and 122 (surrounded completely on all sides by portions of sheath 12). Lumen 114 is identical to lumen 14 of FIGS. 1, 2, and 2A. Lumen 120 is fully enclosed by curved surfaces 140, 144, 148, 150 and planar surfaces 142, 146. These surfaces extend the entire length of sheath 112. The internal walls of sheath 112, including wall 132, provide support for a tube or device being inserted through lumens 114, 120, and 122. Planar surfaces 142, 146 and curved surface 144 are identical to planar surfaces 42, 46 and curved surface 44 of FIG. 1. Curved surface 150 fully encloses lumen 120, instead of having side opening 28 of FIGS. 1-2A. The triangular shape of lumen 120 creates additional space, enabling the physician to insert or utilize therapeutic or diagnostic tools. The internal walls of lumen 120 could be lined with biocompatible materials, such as expanded polytetrafluoroethylene (ePTFE), to reduce friction for any agent or device being inserted through the lumen. The cross-sectional area of lumen 120 accounts for approximately one-fourth (¼) of the cross-sectional area of sheath 112, but that ratio is not limited, as there can be additional or fewer lumens and/or the sizes of lumens can be adjusted. The cross-sectional area of lumen 120 is maximized, to permit agent, for example, to pass therethrough without clogging, while leaving sufficient material of sheath 120 for sufficient structural integrity and passability along a tortuous body lumen. The collective cross-sectional areas of lumens 114, 120, and 122 is at least 50% of the total cross-sectional area of sheath 12, and in certain embodiments about two-thirds to at least about three-fourths of the total cross-sectional area of sheath 112.

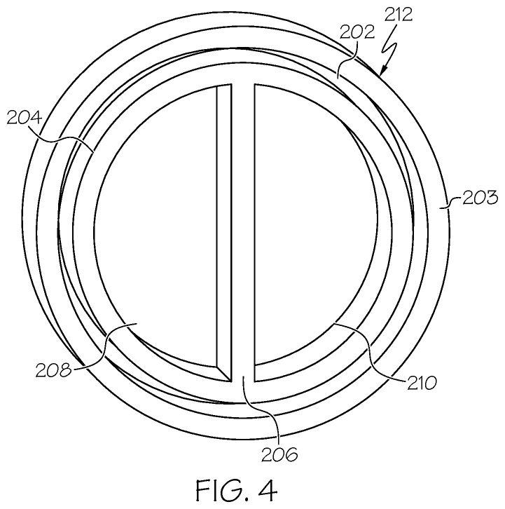

FIG. 4 depicts a cross-sectional view of a portion of a length of exemplary sheath 212. Sheath 212 includes an exterior sheath 203 and an interior sheath 204. Each of exterior sheath 203 and interior sheath 204 can vary in size, shape, and material. For example, for use with endoscopes having a working channel between 2.8 mm and 4.2 mm: the outer diameter of sheath 203 can be between about 0.090″ and 0.155″, the inner diameter of sheath 203 can be between about 0.076″ and 0.141″, the outer diameter of interior sheath 204 can be between about 0.066″ and 0.136″, the area of each of a lumen 208 and a lumen 210 is about 0.0017″ to 0.0062″, or any other dimensions suitable for use with working channels from 2.8 mm to 4.2 mm. The cross-sectional area of lumen 208 accounts for approximately one-third (⅓) of the cross-sectional area of sheath 212, but that ratio is not limited, as there can be additional or fewer lumens and/or the sizes of lumens can be adjusted. The collective cross-sectional areas of lumens 202, 208, and 210 is at least 50% of the total cross-sectional area of sheath 212, and in certain embodiments about two-thirds to at least about three-fourths of the total cross-sectional area of sheath 12. The total usable area of sheath 212 is at least about 25% of the cross-sectional area of the endoscope working channel, but that ratio is not limited, as the sizes of lumens can be adjusted. Additionally, each of sheaths 203, 204 can have a circular outer perimeter and a circular inner perimeter (as shown), but alternatively can have other geometries, including oval, triangles, rectangles, etc. Exterior sheath 203 is independent from interior sheath 204 in that they do not have a structure fixing an outer surface of sheath 204 to an inner surface of sheath 203. In addition, in at least some methods of their use, sheaths 203 and 204 do not contact one another along some or all of the lengths of sheaths 203, 204. The internal surface of exterior sheath 203 and the external surface of interior sheath 204 define an annular lumen 202 that separates sheaths 203, 204. The surfaces of sheaths 203, 204 can be lined with biocompatible materials, such as ePTFE, to reduce friction for any agent or device being inserted through annular lumen 202.

Interior sheath 204 is comprised of two lumens 208, 210 separated by an inter-lumen wall 206. The number, size, and shape of the lumens and inter-lumen walls comprising interior sheath 204 can vary. For example, sheath 204 can define two semicircular shaped lumens (as shown), three triangular-shaped lumens separated by three inter-lumen walls, or more lumens of the same of varying shapes separated by inter-lumen walls. The number of lumens comprising the interior sheath may depend on the material strength and cross-sectional area of interior sheath 204. For example, the material needs to provide sufficient structural support to prevent the sheath from collapsing. Additionally, inter-lumen wall 206 must be of sufficient thickness to enable lumens 208, 210 to retain their shape throughout the length of interior sheath 204, though in some embodiments, inter-lumen wall 206 may be flexible to allow the shape of one or more lumens 208, 210 to accommodate an amount or size of agent(s) or device(s) extending through lumens 208, 210. The lumens within interior sheath 204 can be lined with biocompatible materials, such as ePTFE, to reduce friction for any agent or device being inserted through lumens 208, 210.

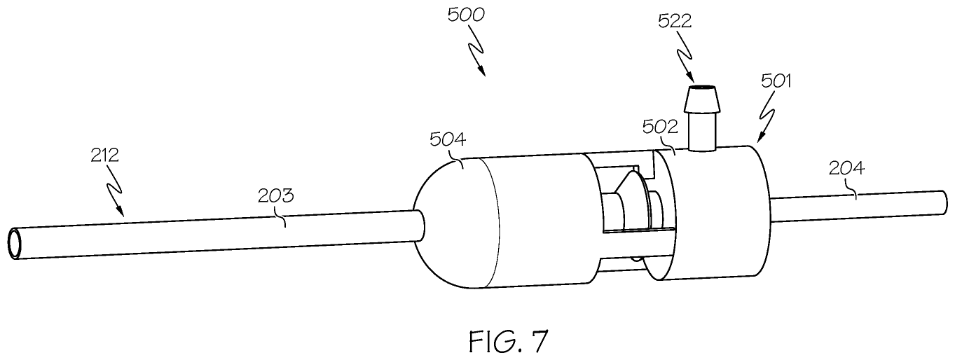

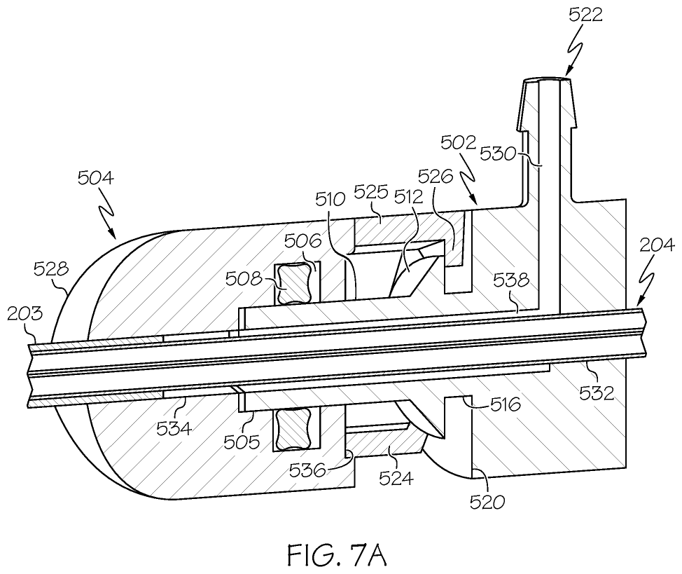

FIG. 7 depicts a perspective view of an exemplary handle 500 to be used with sheath 212. Handle 500 includes handle pieces 502, 504, and a port 522. Handle piece 502 is at or adjacent to the handle proximal end 501. Handle piece 504 is attached to the distal end of handle piece 502.

FIG. 7A provides a cross-sectional view of handle 500. Interior sheath 204 extends from the distal end to the proximal end of handle piece 502 via handle piece lumen 532. Handle piece 502 may be configured to be fixed to the external surface of interior sheath 204 by means of adhesive, a line-to-line fit, an over molding process, or the like. Agent containers can be attached to additional ports affixed to the proximal end of interior sheath 204 by, for example, an adhesive, a heat shrink, a snap-fit connection, a threaded coupling, a luer connection, a crimping connection, or the like. Alternatively, ports can be affixed to the proximal end of handle piece 502 and be in fluid connection with the lumens of interior sheath 204.

Port 522 on handle piece 502 is configured to connect to sources of agents, gas, fluids, or the like, and provides access to annular lumen 202 through port lumen 530. The sources of such agents, gases, or fluids can be any suitable sources known in the art. The longitudinal axis of port lumen 530 is perpendicular to the longitudinal axes of handle piece lumen 532 and interior sheath 204, though that angular relationship is not limited. Handle piece lumen 532 extends from the proximal end to the distal end of handle piece 502. Handle piece lumen 532 is open on the proximal and distal ends. Handle piece 502 can include any actuators known in the art to control the supply of agent, gas, fluid, etc. from port 522 to annular lumen 202. These actuators can include triggers, buttons, switches, pneumatic controls, or other methods known in the art. Alternatively, port 522 and actuators can be configured on handle piece 504 (to be described further herein). Handle piece 502 further includes a collar 510, flange 512, and planar surface 520. These structures assist in connecting handle piece 504 to handle piece 502.

Collar 510 extends distally from a remainder of handle piece 502 and can vary in size and shape. For example, collar 510 can have a cross-sectional shape that is circular, rectangular, or any other desired shape. Collar 510 defines a portion of handle piece lumen 532. Flange 512 is proximal to collar 510 and also defines a portion of handle piece lumen 532. Flange 512 facilitates the attachment of handle piece 504 to handle piece 502. Flange 512 is annular and cone-shaped, and the width dimension tapers in the distal direction. Extension 516 is proximal to flange 512. Extension 516 also defines a portion of handle piece lumen 532 and provides an annular space for snap features 524 of handle piece 504 to lock between flange 512 and the proximal portion of handle piece 502. Planar surface 520 provides a stop for the proximal end of snap features 524.

Handle piece 504 is comprised of distal curved surface 528, handle piece lumen 534, collar cavity 505, seal cavity 506, and snap features 524 each having a projection 526. Curved surface 528 defines the distal end of handle piece 504. Curved surface 528 is not limited to a conical shape, as shown, and can vary in size. For example, the external surface can be rectangular or triangular. Handle piece lumen 534 extends through the center of handle piece 504. Handle piece lumen 534 is open on the proximal and distal ends and communicates with handle piece lumen 532 when handle piece 502 is assembled. Exterior sheath 203 is attached to handle piece lumen 532 using, for example, adhesive, an over molding process, or the like. Collar cavity 505 receives collar 510 of handle piece 502. The size and shape of collar cavity 505 can vary depending on the desired attachment method of handle piece 502. For example, collar cavity 505 can be rectangular and receive a circular collar 510. Alternatively, the collar cavity 505 can be the same geometry as collar 510. These components can be combined together using adhesive, a press-fit method, or the like.

Seal cavity 506 holds seal 508, which may be an 0-ring made of rubber or other suitable material. Seal 508 creates an air-tight seal between collar 510 and collar cavity 505, such that pressurized fluid passing through port lumen 530 into handle piece lumen 534 does not seep out of handle 502. Seal 508 and seal cavity 506 can vary in size and shape.

Snap features 524 extend proximally from a proximally-facing planar surface 536 of handle piece 504. Each snap feature is comprised of a post 525 and a radially-inward projection 526. Post 525 enables the projection to bend and flex radially outward over flange 512. Projection 526 locks in place proximally of flange 512, joining handle piece 504 to handle piece 502. The lock can be permanent or temporary. The size, shape, and number of the snap features 524 are not limited. For example, handle 504 can have two or more snap features 524. Snap features 524 can also vary in size and shape in relation to one another. Snap features 524 further enable handle pieces 502, 504 to rotate relative to one another, but not translate relative to one another. For example, handle piece 502 can rotate clockwise while handle piece 504 is held stationary or rotated counterclockwise, or vice versa. Alternatively, handle pieces 502, 504 can be attached using any other method known in the art, such as a press-fit over collar 510, a cork and screw design, and the like.

The device of FIGS. 7-7A is used in a similar way as device 10 of FIG. 1. To do so, the user first may attach a source of pressurized fluid to port 522 leading to annular lumen 202 and sources of agent to ports leading to the lumens of interior sheath 204. The user then may insert the distal end of sheath 212 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate handle 500 or other actuators coupled to the sources of agent and pressurized fluid (via any known, suitable actuator) to permit agent parts and pressurized fluid to flow through the lumens of sheath 204 and annular space 202 between sheaths 203 and 204. The fluid and agent parts exit the distal end of sheath 212, mix at the target treatment site, and adhere to tissue. The user can rotate handle piece 502 independently of handle piece 504 to manipulate the distal end of the sheath to the desired orientation during a procedure

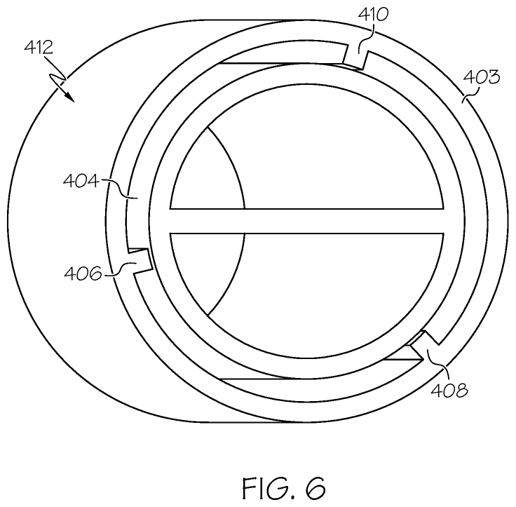

FIG. 6 depicts a cross-sectional view of a portion of a length of an exemplary embodiment of a sheath 412, having an exterior sheath 403 and an interior sheath 404. Interior sheath 404 is identical to interior sheath 204 of FIG. 4. Exterior sheath 403 is identical to exterior sheath 403 of FIG. 4, except as described below. Ribs 406, 408, 410 project radially-inward from the internal surface of exterior sheath 403 and extend a portion of, or the entire length of, exterior sheath 403. Alternatively, ribs 406, 408, and 410 can project from the external surface of interior sheath 404 and extend a portion of, or the entire length of, interior sheath 404. Ribs 406, 408, and 410 provide support to the interior sheath 404. Ribs 406, 408, and 410 also keep the interior sheath 404 spaced from and concentric to exterior sheath 403. The ribs can also alternate from which surface they protrude from. For example, a rib can protrude from the internal surface of the exterior sheath, and a second rib can protrude from the external surface of the interior sheath such that, when the interior sheath 404 and exterior sheath 403 are coupled together, the ribs prevent the interior sheath from freely rotating along the longitudinal axis of the sheath, as the ribs could abut to act as a stop to rotation at a predetermined angle. The embodiment of FIG. 6 can be used with handle 500 (described above).

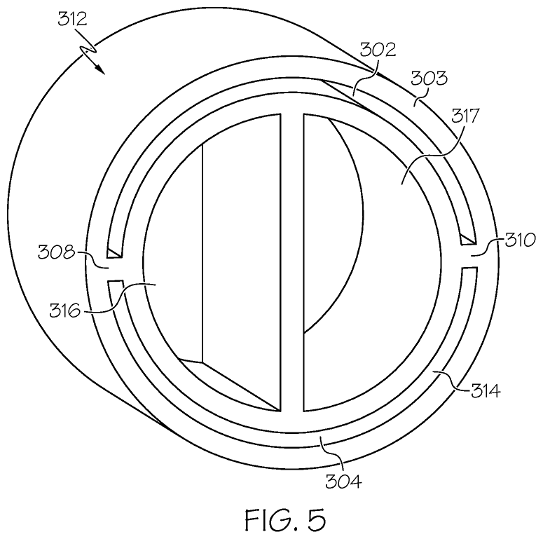

FIG. 5 depicts a cross-sectional view of a portion of a length of an exemplary embodiment of a sheath 312. Sheath 312 is identical to sheath 212 of FIG. 4, except as described below. In sheath 312, the annular lumen between the inner and outer sheaths is separated into two semi-annular lumens 302, 314 by two connectors 308, 310. There can be more than two such connectors, providing for additional such lumens. The semi-annular lumens are separated from lumens 316, 317 by inner sheath 304. This embodiment could be used with handle 500, described above. To do so, a proximal length of exterior sheath 312 can be skived or otherwise removed and attached to handle piece 502, using any of the methods described previously. Handle pieces 502, 504 would not be independently movable relative to each other if sheath 312 is used, however.

Each of the aforementioned systems, devices, assemblies, and methods may be used to protect and/or treat treatment sites by delivering one or more components of an agent to the treatment site. By providing a medical device with multiple lumens for delivering an agent in parts, and mixing the components after dispensing the agents from the lumens, known problems associated with invasive surgical procedures and/or premature curing and clogging of catheters are avoided. Accordingly, physicians may reduce the overall procedure time, increase efficiency of procedures, and/or avoid unnecessary harm to a subject's body caused by limited ability of other tools/devices to treat perforations, post-surgical leaks, or other wounds that might result from endoscopic and open surgical procedures of the gastrointestinal (GI) tract.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only, with a true scope of the disclosure being indicated by the following claims and their equivalents.

We claim:

1. A delivery device, comprising:
a sheath having a proximal end, a distal end, a first channel extending within the sheath from the proximal end to the distal end, and a second channel extending within the sheath from the proximal end to the distal end, wherein a collective cross-sectional area of the first channel and the second channel is more than 50% of a total cross sectional area of the sheath; and
a handle at the proximal end of the sheath, wherein the handle is configured to provide fluid communication between a first port and the first channel and a second port and the second channel.

2. The delivery device of claim 1, wherein the first channel is defined by (1) at least one first planar surface that extends from the proximal end to the distal end of the second channel, and (2) a first side opening that extends from the proximal end to the distal end of the second channel.

3. The delivery device of claim 2, wherein the second channel is defined by (1) at least one second planar surface that extends from the proximal end to the distal end of the second channel, and (2) a second side opening that extends from the proximal end to the distal end of the second channel, wherein the first and second channels are separated by an inter-lumen wall.

4. The delivery device of claim 2, wherein the first side opening separates first and second flanges each configured to flex radially inward and radially outward.

5. The delivery device of claim 2, further comprising a tube within the first channel, wherein a space is defined between the first planar surface and an exterior surface of the tube.

6. The delivery device of claim 1, wherein the sheath is an interior sheath, and the delivery device further comprises:
an exterior sheath surrounding the interior sheath, wherein an at-least partly annular space is defined between the exterior sheath and the interior sheath.

7. The delivery device of claim 6, wherein the exterior sheath rotates relative to the interior sheath.

8. The delivery device of claim 6, wherein the exterior sheath and interior sheath are coupled together by one or more connectors that partially or entirely extend from the proximal end to the distal end of the sheath between an internal surface of the exterior sheath and an external surface of the interior sheath.

9. The delivery device of claim 7, wherein the interior sheath is centered within the exterior sheath by one or more ribs between an internal surface of the exterior sheath and an exterior surface of the interior sheath, such that the at-least partly annular space completely surrounds the interior sheath.

10. The delivery device of claim 9, wherein the ribs alternately protrude from the internal surface of the exterior sheath and the external surface of the interior sheath, such that the rotation of the interior sheath is limited to a predetermined angle.

11. The delivery device of claim 6, wherein the interior sheath defines at least two channels separated by an inter-lumen wall that extends from the proximal end to the distal end of the sheath.

12. The delivery device of claim 6, further comprising a third port of the handle is in fluid communication with the at-least partly annular channel.

13. The delivery device of claim 12, wherein the handle includes a proximal handle piece and a distal handle piece, wherein the interior sheath is fixed to the proximal handle piece, and the exterior sheath is fixed to the distal handle piece, wherein a locking mechanism couples the proximal handle piece to the distal handle piece such that the proximal handle piece rotates relative to the distal handle piece and the interior sheath rotates relative to the exterior sheath.

14. The delivery device of claim 13, wherein the proximal handle piece includes a flange, the distal piece includes one or more protrusions proximal of the flange to couple the proximal handle piece to the distal handle piece, each protrusion is integral with a corresponding flexible post, and each flexible post is configured to bend to extend over the flange, permitting the protrusion to enter a space of the handle proximal to the flange.

15. The delivery device of claim 1, further comprising a third channel extending within the sheath from the proximal end to the distal end, wherein a collective cross-sectional area of the first channel, the second channel, and the third channel is more than 75% of a total cross sectional area of the sheath.

16. A delivery device comprising:
an exterior sheath coupled to a handle;
an interior sheath surrounded by the exterior sheath to define an at-least partly annular channel between the interior sheath and the exterior sheath, wherein the interior sheath is coupled to the handle; and
a port that is in fluid communication with the at-least partly annular lumen.

17. The delivery device of claim 16, wherein an additional port is attached to each of two channels of the interior sheath such that the additional ports are in fluid connection with each respective channels of the interior sheath.

18. The delivery device of claim 16, wherein the handle includes a proximal handle piece and a distal handle piece, wherein the interior sheath is fixed to the proximal handle piece, and the exterior sheath is fixed to the distal handle piece, wherein a locking mechanism couples the proximal handle piece to the distal handle piece such that the proximal handle piece rotates relative to the distal handle piece and the interior sheath rotates relative to the exterior sheath.

19. The delivery device of claim 18, wherein the proximal handle piece includes a flange, and the distal piece includes one or more protrusions proximal of the flange to couple the proximal handle piece to the distal handle piece.

20. A delivery method comprising:
placing a distal end of a delivery device proximate a treatment site; and
at a same time:
deliver a first part of an agent through a first channel of a sheath to proximate the treatment site;
deliver a second part of the agent through a second channel of the sheath to proximate the treatment site;
deliver a pressurized fluid through a third channel of the sheath to proximate the treatment site, wherein the pressurized fluid mixes the first and second parts of the agent for delivery onto the treatment site,
wherein a collective cross-sectional area of the first, second, and third channels is more than 25% of a total cross sectional area of the sheath.
